SEQUENCE LISTING (6 sequences)

SEQ ID NO: 1 - DNA, 1710 bp, Saccharomyces cerevisiae
atgaaggacttgaagttgtctaactttaagggtaaattcatctccagaacctctcactggggtttgactggcaagaaattgagatactttatcaccattgcttctatgactggtttctccttgtttggttacgaccaaggtttgatggcttctctaatcactggcaagcaattcaactacgaatttccagccaccaaggaaaacggtgatcacgacagacatgctaccgtcgttcaaggtgctactacctcctgttacgaattgggttgttttgctggttctttgttcgtcatgttttgcggcgaaagaatcggtagaaagccattgattctaatgggttccgttatcaccattatcggtgctgtcatctctacttgtgcctttcgtggttactgggctttgggtcaattcatcattggcagagttgtcactggtgttggaactggcttgaacacctctactattccagtctggcaatccgaaatgagcaaggccgagaacagaggtttgctagtcaacttggaaggttctactatcgcttttggtaccatgattgcttactggatcgactttggcttgtcctacaccaacagttctgtccaatggagatttccagtttccatgcaaatcgtctttgctttgttcttattggcctttatgatcaagttgccagaatctcctcgttggttgatttctcaaagtcgtaccgaagaggctagatacttggtaggtactttagacgatgccgacccaaacgatgaagaggtcatcaccgaagttgctatgttgcacgacgctgtcaacagaaccaagcacgaaaagcattctttatccagcttgttctccagaggtaggtctcaaaacttgcagagagctttgattgccgcttctactcaattctttcagcaatttactggttgcaacgctgccatctactattctactgtcttgttcaacaagaccatcaagttggactacagattatctatgatcattggtggcgtctttgccactatctacgctttgtccaccatcggttctttctttctaatcgaaaagttgggtagacgtaagctgtttttgttaggtgctactggccaagctgtttccttcaccatcacttttgcctgtttggtcaaggaaaacaaggagaatgctagaggtgccgctgttggtttgttcctgtttatcaccttctttggtttgtctttactatccttgccttggatctacccacccgaaattgcttctatgaaggttcgtgcctccaccaacgctttctctacttgtaccaattggttgtgcaactttgctgttgtcatgtttactccaatcttcattggtcaatctggctggggttgttacttgttctttgccgttatgaattacttgtacattccagtcatcttctttttctacccagaaactgctggtagaagcttggaggaaatcgacattatctttgccaaggcttacgaagatggtactcaaccttggagagttgctaaccacttaccaaagttgtccttgcaagaagtcgaggaccacgccaacgctttgggttcttacgacgatgaaatggagaaggaagactttggtgaagacagagtcgaagatacctacaaccaaatcaatggtgacaactcttccagttcttccaacatcaagaatgaagatactgtcaacgacaaggccaactttgaaggttaa

SEQ ID NO: 2 - protein, 569 aa, Saccharomyces cerevisiae
MKDLKLSNFKGKFISRTSHWGLTGKKLRYFITIASMTGFSLFGYDQGLMASLITGKQFNYEFPATKENGDHDRHATVVQGATTSCYELGCFAGSLFVMFCGERIGRKPLILMGSVITIIGAVISTCAFRGYWALGQFIIGRVVTGVGTGLNTSTIPVWQSEMSKAENRGLLVNLEGSTIAFGTMIAYWIDFGLSYTNSSVQWRFPVSMQIVFALFLLAFMIKLPESPRWLISQSRTEEARYLVGTLDDADPNDEEVITEVAMLHDAVNRTKHEKHSLSSLFSRGRSQNLQRALIAASTQFFQQFTGCNAAIYYSTVLFNKTIKLDYRLSMIIGGVFATIYALSTIGSFFLIEKLGRRKLFLLGATGQAVSFTITFACLVKENKENARGAAVGLFLFITFFGLSLLSLPWIYPPEIASMKVRASTNAFSTCTNWLCNFAVVMFTPIFIGQSGWGCYLFFAVMNYLYIPVIFFFYPETAGRSLEEIDIIFAKAYEDGTQPWRVANHLPKLSLQEVEDHANALGSYDDEMEKEDFGEDRVEDTYNQINGDNSSSSSNIKNEDTVNDKANFEG

SEQ ID NO: 3 - DNA, 1779 bp, Zygosaccharomyces rouxii
atgggtaagagaactcaaggtttcatggactacgtcttttctagaacctccactgctggtttgaagggtgctagattgcgttacactgctgccgctgttgccgtcatcggctttgctttgttcggttacgaccaaggtttgatgtctggtctaatcactggtgatcaattcaacaaggaatttccacctaccaagtccaacggtgacaatgatcgttacgcttctgtcattcaaggtgccgttactgcttgttacgaaatcggctgcttctttggttccttgtttgtcctattctttggtgacgctatcggtagaaagccattgatcattttcggtgctatcattgtcatcattggtaccgttatctctactgcaccatttcaccatgcttggggtttgggccaattcgttgtcggtagagttattactggtgttggtacaggtttcaacacttctaccattccagtgtggcaatctgaaatgacgaaaccaaacatcagaggtgccatgatcaacttggacggttctgtcattgcttttggtactatgatcgcttactggttggacttcggcttttccttcatcaactctagtgttcaatggagatttccagtctctgttcaaatcatttttgccttagtcttgctattcggtatcgtcagaatgccagaatctcccagatggttgatggccaagaaaagaccagcagaagctagatacgtgttggcttgtttgaatgacttaccagaaaacgacgatgccatcttggctgaaatgacttctttgcacgaagctgtcaacagatcctctaaccaaaagtctcaaatgaagtccttgttctctatgggtaagcaacagaacttttccagagccttgattgcttcttccactcaattctttcagcaattcactggttgcaatgctgccatctactattctaccgtcttgtttcaaaccaccgttcaattggacagattactagctatgattttgggtggcgtctttgccactgtttacaccttgtctactttgccatccttctacttagtcgaaaaggttggtagacgtaagatgtttttctttggtgctttgggtcaaggcatctccttcatcattacatttgcttgtttggtcaatccaaccaagcaaaacgccaagggtgctgccgttggtttgtacttattcatcatttgttttggtttggctatcttagaattgccttggatctacccacctgaaattgcttctatgagagttcgtgcagctaccaacgccatgtctacctgtactaactgggttaccaactttgctgttgttatgttcactccagtcttcatccaaacttctcaatggggttgttacttgttctttgctgttatgaacttcatctacttgccagttatctttttcttttacccagaaactgctggtagatccttggaagagatcgacattatctttgccaaggctcacgtggacggtaccttgccttggatggttgctcacagattaccaaagttgtctatgaccgaagttgaggactactcccaatctttgggtctacacgatgacgaaaacgaaaaggaggaatacgacgagaaggaagctgaagccaatgctgccttgtttcaagtcgaaacttcttccaagtctccatcctctaacagaaaggacgatgacgctccaatcgaacataacgaggttcaagaatccaacgacaattcttccaacagctctaacgtcgaagctccaattcctgttcatcacaacgatccataa

SEQ ID NO: 4 - protein, 592 aa, Zygosaccharomyces rouxii
MGKRTQGFMDYVFSRTSTAGLKGARLRYTAAAVAVIGFALFGYDQGLMSGLITGDQFNKEFPPTKSNGDNDRYASVIQGAVTACYEIGCFFGSLFVLFFGDAIGRKPLIIFGAIIVIIGTVISTAPFHHAWGLGQFVVGRVITGVGTGFNTSTIPVWQSEMTKPNIRGAMINLDGSVIAFGTMIAYWLDFGFSFINSSVQWRFPVSVQIIFALVLLFGIVRMPESPRWLMAKKRPAEARYVLACLNDLPENDDAILAEMTSLHEAVNRSSNQKSQMKSLFSMGKQQNFSRALIASSTQFFQQFTGCNAAIYYSTVLFQTTVQLDRLLAMILGGVFATVYTLSTLPSFYLVEKVGRRKMFFFGALGQGISFIITFACLVNPTKQNAKGAAVGLYLFIICFGLAILELPWIYPPEIASMRVRAATNAMSTCTNWVTNFAVVMFTPVFIQTSQWGCYLFFAVMNFIYLPVIFFFYPETAGRSLEEIDIIFAKAHVDGTLPWMVAHRLPKLSMTEVEDYSQSLGLHDDENEKEEYDEKEAEANAALFQVETSSKSPSSNRKDDDAPIEHNEVQESNDNSSNSSNVEAPIPVHHNDP

SEQ ID NO: 5 - DNA, 715 bp, Artificial Sequence
  Synthetic construct
gtcaaaaatagccatcttagcatcgcctgatttggcatcgaccaaaattgcgtcgttttcctttagagaatacttggccaggtattcagccgtgacgtcggcttggaaatctaaaagtgggttacccaatactaccaatggtgcggtcataattgcttgctctttcttttgctgttatctttggttctaccctgcacaagataaactgagatgactacctaattagacatggcatgcctataagtaaagagaattgggctcgaagaataattttcaagcctgccctcatcacgtacgacgacactgcgactcatccatgtgaaaattatcggcatctgcaaaaaaagtttcaacttccacaggtaatattggcatgatgcgaaattggacgtaagtatctctgaagtgcagccgattgggcgtgcgactcacccactcaggacatgatctcagtagcgggttcgataaggcgatgacagcgcaaatgccgcttactggaagtacagaacccgctcccttaggggcacccaccccagcacgccggggggttaaaccggtgtgtcggaattagtaagcggacatcccttccgctgggctcgccatcgcagatatatatataagaagatggttttgggcaaatgtttagctgtaactatgttgcggaaaaacaggcaagaaagcaatcgcgcaaacaaataaaacataattaatttat

SEQ ID NO: 6 - protein, 1173 aa, Artificial Sequence
  Synthetic construct
MTSPVIGTPWKKLNAPVSEAAIEGVDKYWRVANYLSIGQIYLRSNPLMKEPFTREDVKHRLVGHWGTTPGLNFLIGHINRFIAEHQQNTVIIMGPGHGGPAGTAQSYLDGTYTEYYPKITKDEAGLQKFFRQFSYPGGIPSHFAPETPGSIHEGGELGYALSHAYGAVMNNPSLFVPAIVGDGEAETGPLATGWQSNKLVNPRTDGIVLPILHLNGYKIANPTILSRISDEELHEFFHGMGYEPYEFVAGFDDEDHMSIHRRFADMFETIFDEICDIKAEAQTNDVTRPFYPMIIFRTPKGWTCPKFIDGKKTEGSWRAHQVPLASARDTEAHFEVLKNWLKSYKPEELFNEDGSIKEDVLSFMPQGELRIGQNPNANGGRIREDLKLPNLDDYEVKEVKEFGHGWGQLEATRRLGVYTRDVIKNNPDSFRIFGPDETASNRLQAAYEVTNKQWDAGYLSELVDEHMAVTGQVTEQLSEHQMEGFLEAYLLTGRHGIWSSYESFVHVIDSMLNQHAKWLEATVREIPWRKPISSMNLLVSSHVWRQDHNGFSHQDPGVTSVLLNKTFNNDHVIGIYFPVDSNMLLAVGEKVYKSTNMINAIFAGKQPAATWLTLDEAREELEKGAAEWKWASNAKNNDEVQVVLAGIGDVPQQELMAAADKLNKLGVKFKVVNIVDLLKLQSAKENNEALTDEEFTELFTADKPVLLAYHSYAHDVRGLIFDRPNHDNFNVHGYKEQGSTTTPYDMVRVNDMDRYELTAEALRMVDADKYADEIKKLEDFRLEAFQFAVDKGYDHPDYTDWVWPGVKTDKPGAVTATAATAGDNEGAGPARPAGLPPATYYDSLAVTSMDLFESLAQKITGKDQTIVFPEGTEPRIVGAAARLAADGLVKPIVLGATDKVQAVANDLNADLTGVQVLDPATYPAEDKQAMLDALVERRKGKNTPEQAAKMLEDENYFGTMLVYMGKADGMVSGAIHPTGDTVRPALQIIKTKPGSHRISGAFIMQKGEERYVFADCAINIDPDADTLAEIATQSAATAKVFDIDPKVAMLSFSTKGSAKGEMVTKVQEATAKAQAAEPELAIDGELQFDAAFVEKVGLQKAPGSKVAGHANVFVFPELQSGNIGYKIAQRFGHFEAVGPVLQGLNKPVSDLSRGCSEEDVYKVAIITAAQGLA

(19) United States
(12) Patent Application Publication
(10) Pub. No.: US 2020/0377559 A1
(43) Pub. Date: Dec. 3, 2020

(54) REDUCTION OF ACETATE AND GLYCEROL IN MODIFIED YEAST HAVING AN EXOGENOUS ETHANOL-PRODUCING PATHWAY

(71) Applicant: DANISCO US INC, Palo Alto, CA (US)

(72) Inventors: Daniel Joseph Macool, Rutledge, PA (US); Paula Johanna Maria Teunissen, San Jose, CA (US); Yehong Jamie Wang, Wilmington, DE (US); Hyeryoung Yoon, Newark, DE (US); Quinn Qun Zhu, West Chester, PA (US)

(21) Appl. No.: 16/497,236

(22) PCT Filed: Mar. 24, 2018
(86) PCT No.: PCT/US18/24222

§ 371 Date: Sep. 24, 2019

Related U.S. Application Data
(60) Provisional application No. 62/476,436, filed on Mar. 24, 2017.
(60) Provisional application No. 62/520,596, filed on Jun. 16, 2017.

(51) Int. Cl. C07K 14/395; C12N 15/81

(52) U.S. Cl. CPC C07K 14/395; C12N 15/81

(57) ABSTRACT

Described are compositions and methods relating to the over-expression of sugar transporter-like polypeptides to reduce the amount of glycerol and acetate produced by modified yeast having an exogenous pathway that cause it to produce more ethanol and acetate than its parental yeast.

### PRIORITY

The present application claims the benefit of U.S. Provisional Application Ser. Nos. 62/476,436, filed Mar. 24, 2017, and 62/520,596, filed Jun. 16, 2017, each of which is hereby incorporated by reference in its entirety.

### TECHNICAL FIELD

The present compositions and methods relate to the over-expression of sugar transporter-like polypeptides to reduce the amount of glycerol and acetate produced by modified yeast having an exogenous pathway that cause it to produce more ethanol and acetate than its parental yeast.

### BACKGROUND

The first generation of yeast-based ethanol production converts sugars into fuel ethanol. The annual fuel ethanol production by yeast is about 90 billion liters worldwide (Gombert, A. K. and van Maris. A. J. (2015) Curr Opin Biotechnol. 33:81-86). It is estimated that about 70% of the cost of ethanol production is the feedstock. Since the production volume is so large, even small yield improvements will have massive economic impact across the industry.

From a biochemical perspective, the conversion of one mole of glucose into two moles of ethanol and two moles of carbon dioxide is redox-neutral with a maximum theoretical yield of about 51% (wt/wt). The current industrial yield is about 45%, and the yeast accumulates a surplus of NADH that is used to produce glycerol for redox balance and osmotic protection. There is, therefore, opportunity to increase ethanol production yield by about 10%, which translates into an extra nine billion liters of ethanol per year.

Aside from the production of carbon dioxide, yeast biomass and glycerol are the two major by-products of the fermentation process. Glycerol, a small, uncharged molecule, is the main and most frequently used osmotic protectant in yeast (Duškova, M. et al. (2015) Mol Microbiol. 97:541-59). There is about 10-15 g/L glycerol and about 5 g/L yeast biomass produced in current industrial corn mash fermentation. It has been estimated that about 5 g/L glycerol, at a 1:1 ratio to biomass, is needed to balance the surplus NADH generated from biosynthetic reactions.

Several strategies, such as the knock-out or down regulation of glycerol biosynthetic genes encoding glycerol-3-phosphate dehydrogenase (i.e., GPD1 and GPD2), have been tried to eliminate or reduce the glycerol production. Deletion of both GPD1 and GPD2 genes eliminated glycerol production but the modified yeast was unable to grow under anaerobic conditions (Björkqvist, S. et al. (1997) Appl Environ Microbiol. 63:128-132). Fine-turning of the promoter strengths of GPD1 and GPD2 reduced the amount of glycerol but the resulting strains were not sufficiently robust for industrial applications (Pagliardini, J. et al. (2013) Microbial Cell Factories. 12:29).

Yeast has a complex system for controlling glycerol transportation. Glycerol is exported from the cell by means of FPS1, an aquaporin channel protein belonging to the family of major intrinsic proteins. To increase the amount of intracellular glycerol, the FPS1 channel remains closed under hyperosmotic conditions (Remize, F. et al. (2001) Metab Eng. 3:301-312). Glycerol is imported into the cell via the sugar transporter-like (STL) transporter, STL1. This transporter is structurally related to the family of hexose transporters within the major facilitator superfamily. STL1 is involved with the uptake of glycerol at the expense of ATP (Ferreira, C. et al. (2005) Mol Biol Cell. 16:2068-76; Dušková et al., 2015).

The glycerol import function of STLs from Saccharomyces cerevisiae (Ferreira et al., 2005), Candida albicans (Kayingo, G. et al. (2009)Microbiology. 155:1547-57), Pichia sorbitophila (WO 2015023989 A1), Zygosaccharomyces rouxii (Duškovä et al., 2015) have been described, and the STL1 of P. sorbitophila has been used to reduce glycerol in genetically-modified yeast strains (WO 2015023989 A1).

Introduction of components of an exogenous phosphoketolase (PKL) pathway has been used to modify yeast to produce more ethanol and reduced glycerol (Sonderegger, M. et al. (2004) Appl Environ Microbiol. 70:2892-97; Miasnikov et al. (2015) WO 2015/148272 A1). However, the engineered strains also produced more acetate byproduct compared to the parental strains. Acetate is not only a “waste” of carbon, it also adversely affects yeast growth and ability to produce ethanol, particularly under the low pH conditions used in ethanol production facilities to avoid unwanted microbial contamination.

The ongoing need exists to reduce the amount of acetate produced by modified yeast to realize the full potential of increased ethanol production that can be made possible from yeast pathway engineering.

### SUMMARY

The present compositions and methods relate to the over-expression of sugar transporter-like polypeptides in modified yeast having an exogenous pathway that results in the production of more ethanol and acetate than is produced by the parental yeast. Aspects and embodiments of the compositions and methods are described in the following, independently-numbered paragraphs.

1. In one aspect, a method for decreasing the production of glycerol and acetate in cells grown on a carbohydrate substrate is provided, comprising: introducing into modified yeast comprising an exogenous pathway that causes it to produce more ethanol and acetate than its parental yeast a genetic alteration that increases the production of STL1 polypeptides compared to the amount produced in the parental yeast.

2. In some embodiments of the method of paragraph 1, the genetic alteration comprises introducing an expression cassette for expressing an STL1 polypeptide.

3. In some embodiments of the method of paragraph 1, the genetic alteration comprises introducing an exogenous gene encoding an STL1 polypeptide.

4. In some embodiments of the method of paragraph 1, the genetic alteration comprises introducing a stronger or regulated promoter in an endogenous gene encoding an STL1 polypeptide.

5. In some embodiments of the method of any of paragraphs 1-4, the decrease in production of acetate is at least 10% compared to the production by the parental cells grown under equivalent conditions.

6. In some embodiments of the method of any of paragraphs 1-5, the decrease in production of acetate is at least 15% compared to the production by the parental cells grown under equivalent conditions.

7. In some embodiments of the method of any of paragraphs 1-6, the exogenous pathway is the phosphoketolase pathway.

8. In some embodiments of the method of paragraph 7, the phosphoketolase pathway includes a phosphoketolase enzyme and a phosphotransacetylase enzyme.

9. In some embodiments of the method of paragraph 8, the phosphoketolase and phosphotransacetylase are in the form of a fusion polypeptide.

10. In some embodiments of the method of any of paragraphs 1-9, the cells further comprise an exogenous gene encoding a carbohydrate processing enzyme.

11. In some embodiments of the method of paragraph 10, the carbohydrate processing enzyme is a glucoamylase or an alpha-amylase.

12. In some embodiments of the method of any of paragraphs 1-11, the cells further comprise an alteration in the glycerol pathway and/or the acetyl-CoA pathway.

13. In some embodiments of the method of any of paragraphs 1-12, the cells are of a Saccharomyces spp.

These and other aspects and embodiments of present modified cells and methods will be apparent from the description, including the accompanying Figures.

### BRIEF DESCRIPTION OF THE DRAWINGS

FIG. 1 is a diagram of the engineered phosphoketolase pathway for producing ethanol and acetate from sugars.

FIG. 2 is a map of plasmid pZK41Wn.

FIG. 3 is a map of the SwaI fragment from plasmid pZK41Wn-DScSTL.

FIG. 4 is a map of the SwaI fragment from plasmid pZK41Wn-DZrSTL.

FIG. 5 is a map of the SwaI fragment from plasmid pZK41Wn.

FIG. 6 is a map of plasmid pZK41W-GLAF12.

FIG. 7 is a map of plasmid pTOPO II-Blunt ura3-loxP-KanMX-loxP-ura3.

FIG. 8 is a map of the EcoRI fragment from plasmid pTOPO II-Blunt ura3-loxP-KanMX-loxP-ura3.

FIG. 9 is a map of plasmid pGAL-Cre-316.

FIG. 10 is a map of the SwaI fragment from plasmid pZK41W-GLAF12.

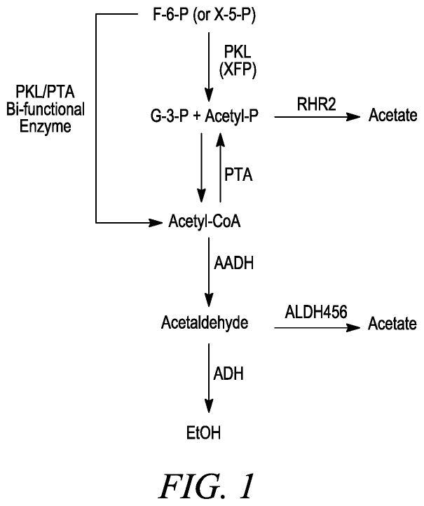

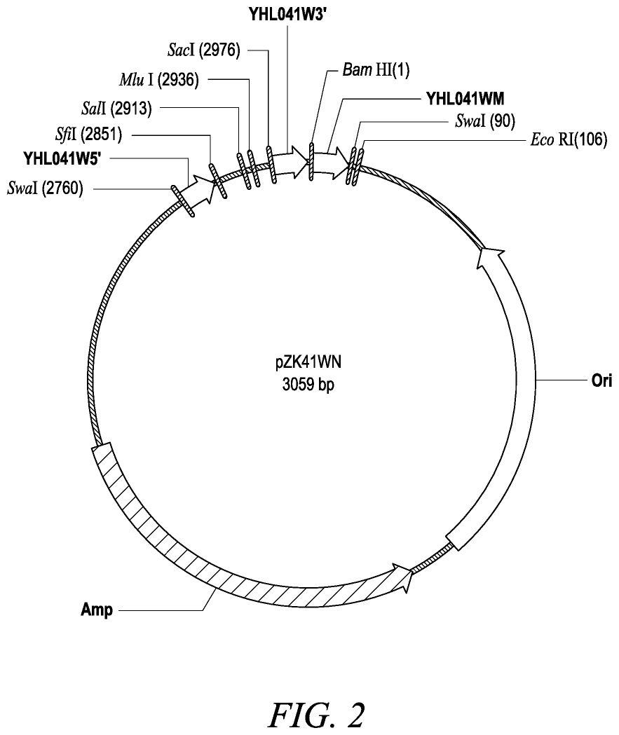

### DETAILED DESCRIPTION

### I. Definitions

Prior to describing the present yeast strains and methods in detail, the following terms are defined for clarity. Terms not defined should be accorded their ordinary meanings as used in the relevant art.

As used herein, “alcohol” refer to an organic compound in which a hydroxyl functional group (—OH) is bound to a saturated carbon atom.

As used herein, the terms “yeast cells,” yeast strains,” or simply “yeast” refer to organisms from the phyla Ascomycota and Basidiomycota. Exemplary yeast is budding yeast from the order Saccharomycetales. Particular examples of yeast are Saccharomyces spp., including but not limited to S. cerevisiae. Yeast include organisms used for the production of fuel alcohol as well as organisms used for the production of potable alcohol, including specialty and proprietary yeast strains used to make distinctive-tasting beers, wines, and other fermented beverages.

As used herein, the phrase “engineered yeast cells,” “variant yeast cells,” “modified yeast cells,” or similar phrases, refer to yeast that include genetic modifications and characteristics described herein. Variant/modified yeast do not include naturally occurring yeast.

As used herein, the terms “polypeptide” and “protein” (and their respective plural forms) are used interchangeably to refer to polymers of any length comprising amino acid residues linked by peptide bonds. The conventional one-letter or three-letter codes for amino acid residues are used herein and all sequence are presented from an N-terminal to C-terminal direction. The polymer can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

As used herein, functionally and/or structurally similar proteins are considered to be “related proteins”, or “homologs”. Such proteins can be derived from organisms of different genera and/or species, or different classes of organisms (e.g., bacteria and fungi), or artificially designed. Related proteins also encompass homologs determined by primary sequence analysis, determined by secondary or tertiary structure analysis, or determined by immunological cross-reactivity, or determined by their functions.

As used herein, the term “homologous protein” refers to a protein that has similar activity and/or structure to a reference protein. It is not intended that homologs necessarily be evolutionarily related. Thus, it is intended that the term encompass the same, similar, or corresponding enzyme(s) (i.e., in terms of structure and function) obtained from different organisms. In some embodiments, it is desirable to identify a homolog that has a quaternary, tertiary and/or primary structure similar to the reference protein. In some embodiments, homologous proteins induce similar immunological response(s) as a reference protein. In some embodiments, homologous proteins are engineered to produce enzymes with desired activity(ies).

The degree of homology between sequences can be determined using any suitable method known in the art (see, e.g., Smith and Waterman (1981) Adv. Appl. Math. 2:482; Needleman and Wunsch (1970) J Mol. Biol., 48:443; Pearson and Lipman (1988) Proc. Natl. Acad. Sci. USA 85:2444; programs such as GAP, BESTFIT, FASTA, and TFASTA in the Wisconsin Genetics Software Package (Genetics Computer Group, Madison, Wis.); and Devereux et al. (1984) Nucleic Acids Res. 12:387-95).

For example, PILEUP is a useful program to determine sequence homology levels. PILEUP creates a multiple sequence alignment from a group of related sequences using progressive, pair-wise alignments. It can also plot a tree showing the clustering relationships used to create the alignment. PILEUP uses a simplification of the progressive alignment method of Feng and Doolittle, (Feng and Doolittle (1987) J Mol. Evol. 35:351-60). The method is similar to that described by Higgins and Sharp ((1989) CABIOS 5:151-53). Useful

PILEUP parameters including a default gap weight of 3.00, a default gap length weight of 0.10, and weighted end gaps. Another example of a useful algorithm is the BLAST algorithm, described by Altschul et al. ((1990) J Mol. Biol. 215:403-10) and Karlin et al. ((1993) Proc. Natl. Acad. Sci. USA 90:5873-87). One particularly useful BLAST program is the WU-BLAST-2 program (see, e.g., Altschul et al. (1996) Meth. Enzymol. 266:460-80). Parameters “W,” “T,” and “X” determine the sensitivity and speed of the alignment. The BLAST program uses as defaults a word-length (W) of 11, the BLOSUM62 scoring matrix (see, e.g., Henikoff and Henikoff (1989) Proc. Natl. Acad. Sci. USA 89:10915) alignments (B) of 50, expectation (E) of 10, M′5, N′-4, and a comparison of both strands.

As used herein, the phrases “substantially similar” and “substantially identical,” in the context of at least two nucleic acids or polypeptides, typically means that a polynucleotide or polypeptide comprises a sequence that has at least about 70% identity, at least about 75% identity, at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, or even at least about 99% identity, or more, compared to the reference (i.e., wild-type) sequence. Percent sequence identity is calculated using CLUSTAL W algorithm with default parameters. See Thompson et al. (1994) Nucleic Acids Res. 22:4673-4680. Default parameters for the CLUSTAL W algorithm are:

| | |
|---|---|
| Gap opening penalty: | 10.0 |
| Gap extension penalty: | 0.05 |
| Protein weight matrix: | BLOSUM series |
| DNA weight matrix: | IUB |
| Delay divergent sequences %: | 40 |
| Gap separation distance: | 8 |
| DNA transitions weight: | 0.50 |
| List hydrophilic residues: | GPSNDQEKR |
| Use negative matrix: | OFF |
| Toggle Residue specific penalties: | ON |
| Toggle hydrophilic penalties: | ON |
| Toggle end gap separation penalty | OFF |

Another indication that two polypeptides are substantially identical is that the first polypeptide is immunologically cross-reactive with the second polypeptide. Typically, polypeptides that differ by conservative amino acid substitutions are immunologically cross-reactive. Thus, a polypeptide is substantially identical to a second polypeptide, for example, where the two peptides differ only by a conservative substitution. Another indication that two nucleic acid sequences are substantially identical is that the two molecules hybridize to each other under stringent conditions (e.g., within a range of medium to high stringency).

As used herein, the term “gene” is synonymous with the term “allele” in referring to a nucleic acid that encodes and directs the expression of a protein or RNA. Vegetative forms of filamentous fungi are generally haploid, therefore a single copy of a specified gene (i.e., a single allele) is sufficient to confer a specified phenotype.

As used herein, the term “expressing a polypeptide” and similar terms refers to the cellular process of producing a polypeptide using the translation machinery (e.g., ribosomes) of the cell.

As used herein, “overexpressing a polypeptide,” “increasing the expression of a polypeptide,” and similar terms, refer to expressing a polypeptide at higher-than-normal levels compared to those observed with parental or “wild-type cells that do not include a specified genetic modification.

As used herein, an “expression cassette” refers to a nucleic acid that includes an amino acid coding sequence, promoters, terminators, and other nucleic acid sequence needed to allow the encoded polypeptide to be produced in a cell. Expression cassettes can be exogenous (i.e., introduced into a cell) or endogenous (i.e., extant in a cell).

As used herein, the terms “fused” and “fusion” with respect to two polypeptides refer to a physical linkage causing the polypeptide to become a single molecule.

As used herein, the terms “wild-type” and “native” are used interchangeably and refer to genes, proteins or strains found in nature.

As used herein, the term “protein of interest” refers to a polypeptide that is desired to be expressed in modified yeast. Such a protein can be an enzyme, a substrate-binding protein, a surface-active protein, a structural protein, a selectable marker, or the like, and can be expressed at high levels. The protein of interest is encoded by a modified endogenous gene or a heterologous gene (i.e., gene of interest”) relative to the parental strain. The protein of interest can be expressed intracellularly or as a secreted protein.

As used herein, “deletion of a gene,” refers to its removal from the genome of a host cell. Where a gene includes control elements (e.g., enhancer elements) that are not located immediately adjacent to the coding sequence of a gene, deletion of a gene refers to the deletion of the coding sequence, and optionally adjacent enhancer elements, including but not limited to, for example, promoter and/or terminator sequences, but does not require the deletion of non-adjacent control elements. The “deletion of a gene” also refers to its functional remove from the genome of a host cell.

As used herein, “disruption of a gene” refers broadly to any genetic or chemical manipulation, i.e., mutation, that substantially prevents a cell from producing a function gene product, e.g., a protein, in a host cell. Exemplary methods of disruption include complete or partial deletion of any portion of a gene, including a polypeptide-coding sequence, a promoter, an enhancer, or another regulatory element, or mutagenesis of the same, where mutagenesis encompasses substitutions, insertions, deletions, inversions, and combinations and variations, thereof, any of which mutations substantially prevent the production of a function gene product. A gene can also be disrupted using RNAi, antisense, Cas9-mediated technology or any other method that abolishes gene expression. A gene can be disrupted by deletion or genetic manipulation of non-adjacent control elements.

As used herein, the terms “genetic manipulation” and “genetic alteration” are used interchangeably and refer to the alteration/change of a nucleic acid sequence. The alteration can include but is not limited to a substitution, deletion, insertion or chemical modification of at least one nucleic acid in the nucleic acid sequence.

As used herein, a “functional polypeptide/protein” is a protein that possesses an activity, such as an enzymatic activity, a binding activity, a surface-active property, or the like, and which has not been mutagenized, truncated, or otherwise modified to abolish or reduce that activity. Functional polypeptides can be thermostable or thermolabile, as specified.

As used herein, “a functional gene” is a gene capable of being used by cellular components to produce an active gene product, typically a protein. Functional genes are the antithesis of disrupted genes, which are modified such that they cannot be used by cellular components to produce an active gene product, or have a reduced ability to be used by cellular components to produce an active gene product.

As used herein, yeast cells have been “modified to prevent the production of a specified protein” if they have been genetically or chemically altered to prevent the production of a functional protein/polypeptide that exhibits an activity characteristic of the wild-type protein. Such modifications include, but are not limited to, deletion or disruption of the gene encoding the protein (as described, herein), modification of the gene such that the encoded polypeptide lacks the aforementioned activity, modification of the gene to affect post-translational processing or stability, and combinations, thereof.

As used herein, “attenuation of a pathway” or “attenuation of the flux through a pathway” i.e., a biochemical pathway, refers broadly to any genetic or chemical manipulation that reduces or completely stops the flux of biochemical substrates or intermediates through a metabolic pathway. Attenuation of a pathway may be achieved by a variety of well-known methods. Such methods include but are not limited to: complete or partial deletion of one or more genes, replacing wild-type alleles of these genes with mutant forms encoding enzymes with reduced catalytic activity or increased Km values, modifying the promoters or other regulatory elements that control the expression of one or more genes, engineering the enzymes or the mRNA encoding these enzymes for a decreased stability, misdirecting enzymes to cellular compartments where they are less likely to interact with substrate and intermediates, the use of interfering RNA, and the like.

As used herein, “aerobic fermentation” refers to growth in the presence of oxygen.

As used herein, “anaerobic fermentation” refers to growth in the absence of oxygen.

As used herein, the singular articles “a,” “an,” and “the” encompass the plural referents unless the context clearly dictates otherwise. All references cited herein are hereby incorporated by reference in their entirety. The following abbreviations/acronyms have the following meanings unless otherwise specified:
EC enzyme commission
PKL phosphoketolase
PTA phosphotransacetylase
XFP xylulose 5-phosphate/fructose 6-phosphate phosphoketolase
AADH acetaldehyde dehydrogenases
ADH alcohol dehydrogenase
EtOH ethanol
AA a-amylase
GA glucoamylase
° C. degrees Centigrade
bp base pairs
DNA deoxyribonucleic acid
ds or DS dry solids
g or gm gram
g/L grams per liter
GAU/g ds glucoamylase units per gram dry solids
H₂O water
HPLC high performance liquid chromatography
hr or h hour
kg kilogram
M molar
mg milligram
mL or ml milliliter
min minute
mM millimolar
N normal
nm nanometer
PCR polymerase chain reaction
ppm parts per million
Δ relating to a deletion
μ microgram
μL nad μl microliter
μM micromolar

### II. Modified Yeast Cells Overexpressing Sugar Transporter-Like Proteins

The present inventors have discovered that over-expression of sugar transporter-like (STL1) polypeptide in yeast simultaneously reduces both glycerol and acetate production in modified yeast having an exogenous pathway that causes it to produce more ethanol and acetate compared to its parental yeast. While expression of STL1 has previously been associated with glycerol reduction (Ferreira et al., 2005; Dušková et al., 2015 and WO 2015023989 A1), it was heretofore unknown that over-expression of STL1 reduces the production of not only glycerol, but also acetate. Reduction in acetate is highly desirable, particularly in cells with an exogenous pathway that causes it to produce more acetate than its parental yeast, such as an exogenous phosphoketolase (PKL) pathway.

The experimental data provided herein demonstrate that the introduction of exogenous, codon-optimized polynucleotides encoding STL1 derived from both S. cerevisiae and Z. rouxii (previously described by Ferreira et al., 2005; Dušková et al., 2015, respectively) reduce acetate production compared to that of parental yeast. Amino acid sequence comparisons showed that there is only about 63% amino acid sequence identity between STL1 derived from Saccharomyces cerevisiae (ScSTL; (SEQ ID NO: 2) and Zygosaccharomyces rouxii (ZrSTL; (SEQ ID NO: 4). Accordingly, it is believed that overexpression of other STL1 are likely to provide similar benefits to yeast, and the present compositions and methods are not limited to particular STL1. STL1 likely to function according to the present compositions and methods are listed in Table 1, where amino acid sequence identity to ScSTL and ZrSTL is provided.

TABLE 1
STL1 from public databases

| | | % Identity with | GenBank |
|---|---|---|---|
| Gene Name | Source organism | ScSTL/ZrSTL | Accession #s |
| ScSTL1 | S. cerevisiae | 100%/63.4% | AAB64975 |
| ZrSTL1 | Z. rouxii | 63.4%/100% | GAV49403 |
| AaSTL1 | Aspergillus aculeatus | 53.9%/51.3% | OJJ99073 |
| AtSTL1 | Aspergillus terreus | 53.7%/54.6% | XP_001209239 |
| BbSTL1 | Brettanomyces bruxellensis | 55.8%/54.6% | AGR86104 |
| CalSTL1 | Candida albicans | 60.5%/64% | XP_718089 |
| CarSTL1 | Candida arabinofermentans | 61.7%/58.6% | ODV84200 |
| CdSTL1 | Candida dubliniensis | 60.3%/62.1% | XP_002421142 |
| CiSTL1 | Candida intermedia | 62.3%/60.3% | SGZ53333 |
| ClSTL1 | Clavispora lusitaniae | 63.9%/61.2% | XP_002619861 |
| CmSTL1 | Candida maltosa | 63.1%/64.6% | EMG50229 |
| CoSTL1 | Candida orthopsilosis | 61.2%/63.5% | XP_003871470 |
| CpSTL1 | Candida parapsilosis | 59.2%/61.1% | CCE39633 |
| CtaSTL1 | Candida tanzawaensis | 61.8%/60.0% | ODV77260 |
| CteSTL1 | Candida] tenuis | 59.2%/60.0% | XP_006687420 |
| CtrSTL1 | Candida tropicalis | 62.8%/60.5% | XP_002551118 |
| DfSTL1 | Debaryomyces fabryi | 59.0%/61.5% | XP_015467278 |
| DhSTL1A | Debaryomyces hansenii | 56.2%/62.3% | XP_459386 |
| DhSTL1B | Debaryomyces hansenii | 61.9%/59.2% | XP_459387 |
| DhSTL1C | Debaryomyces hansenii | 59.5%/61.7% | XP_457182 |
| EcSTL1 | Eremothecium cymbalariae | 64.9%/60.7% | XP_003645723 |
| EgSTL1 | Eremothecium gossypii | 68.5%/63.8% | NP_984235 |
| EsSTL1 | Eremothecium sinecaudum | 63.4%/61.0% | XP_017987889 |
| HbSTL1 | Hyphopichia burtonii | 56.8%/57.2% | DV64743 |
| KbSTL1 | Kalmanozyma brasiliensis | 58.3%/56.2% | XP_016293550 |
| KdSTL1 | Kluyveromyces dobzhanskii | 69.8%/62.9% | CDO96534 |
| KlSTL1 | Kluyveromyces lactis | 69.1%/63.3% | XP_456249 |
| KmSTL1 | Kluyveromyces marxianus | 68.4%/61.7% | BAO41471 |
| LdSTL1 | Lachancea dasiensis | 70.2%/64.0% | SCU85709 |
| LeSTL1 | Lodderomyces elongisporus | 60.7%/58.5% | XP_001524136 |
| LfSTL1 | Lachancea fermentati | 69.2%/64.8% | SCW03899 |
| LlTL1 | Lachancea lanzarotensis | 69.9%/61.8% | CEP62795 |
| LmSTL1 | Lachancea meyersii | 70.5%/60% | SCU83135 |
| LnSTL1 | Lachancea nothofagi | 68.3%/61.9% | SCU96367 |
| LqSTL1 | Lachancea quebecensis | 67.0%/64.1% | CUS22279 |
| LtSTL1 | Lachancea thermotolerans | 66.8%/63.7% | XP_002551983 |
| MaSTL1 | Moesziomyces aphidis | 55.0%/56.9% | ETS61600 |
| MbSTL1 | Metschnikowia bicuspidata | 62.5%/62.0% | XP_018712535 |
| MfSTL1A | Millerozyma farinosa | 59.8%/61.0% | XP_004204749 |
| MfSTL1B | Millerozyma farinosa | 58.4%/59.7% | XP_004204191 |
| MgSTL1 | Meyerozyma guilliermondii | 60.7%/63.0% | XP_001483277 |
| OpSTL1 | Ogataea parapolymorpha | 56.8%/55.1% | XP_013934782 |
| OpoSTL1 | Ogataea polymorpha | 57.0%/54.5% | XP_018211084 |
| PkSTL1 | Pichia kudriavzevii | 57.0%/54.4% | KGK37649 |
| PmSTL1 | Pichia membranifaciens | 58.2%/56.9% | XP_019015383 |
| SaSTL1 | Saccharomyces arboricola | 90.2%/63.6% | EJS42123 |
| SeSTL1_{—} | Saccharomyces eubayanus | 92.0%/62.3% | XP_018220374 |
| SlSTL1 | Sugiyamaella lignohabitans | 58.3%/63.4% | XP_018733704 |
| SsSTL1 | Saccharomycetaceae sp. | 68.8%/63.4% | AGO11904 |
| SstSTL1 | Scheffersomyces stipitis | 61.2%/60.9% | XP_001383774 |
| TdSTL1 | Torulaspora delbrueckii | 74.8%/63.4% | XP_003680062 |
| WaSTL1 | Wickerhamomyces anomalus | 57.1%/60.5% | XP_019036641 |
| WcSTL1 | Zygosaccharomyces bailii | 56.4%/57.3% | XP_011274863 |
| ZbSTL1 | Zygosaccharomyces bailii | 63.6%/81.4% | CDH12218 |

STL1 polypeptides that are expected to work as described, include those having at least 51%, at least 54%, at least 57%, 60%, at least 63%, at least 65%, at least 70%, at least 80%, at least 90%, at least 95%, at least 97%, at least 98%, at least 99%, or more amino acid sequence identity to ScSTL and/or ZrSTL, and/or structural and functional homologs and related proteins. In some embodiments, STL1 polypeptides include substitutions that do not substantially affect the structure and/or function of the polypeptide. Exemplary substitutions are conservative mutations, as summarized in Table 2.

TABLE 2
Exemplary amino acid substitutions

| Original Amino | | |
|---|---|---|
| Acid Residue | Code | Acceptable Substitutions |
| Alanine | A | D-Ala, Gly, β-Ala, L-Cys, D-Cys |
| Arginine | R | D-Arg, Lys, D-Lys, homo-Arg, |
| | | D-homo-Arg, Met, Ile, |
| | | D-Met, D-Ile, Orn, D-Orn |
| Asparagine | N | D-Asn, Asp, D-Asp, Glu, D-Glu, |
| | | Gln, D-Gln |
| Aspartic Acid | D | D-Asp, D-Asn, Asn, Glu, D-Glu, |
| | | Gln, D-Gln |
| Cysteine | C | D-Cys, S-Me-Cys, Met, D-Met, |
| | | Thr, D-Thr |
| Glutamine | Q | D-Gln, Asn, D-Asn, Glu, D-Glu, |
| | | Asp, D-Asp |
| Glutamic Acid | E | D-Glu, D-Asp, Asp, Asn, D-Asn, |
| | | Gln, D-Gln |
| Glycine | G | Ala, D-Ala, Pro, D-Pro, β-Ala, |
| | | Acp |
| Isoleucine | I | D-Ile, Val, D-Val, Leu, D-Leu, |
| | | Met, D-Met |
| Leucine | L | D-Leu, Val, D-Val, Leu, D-Leu, |
| | | Met, D-Met |
| Lysine | K | D-Lys, Arg, D-Arg, homo-Arg, |
| | | D-homo-Arg, Met, D-Met, |
| | | Ile, D-Ile, Orn, D-Orn |
| Methionine | M | D-Met, S-Me-Cys, Ile, D-Ile, |
| | | Leu, D-Leu, Val, D-Val |
| Phenylalanine | F | D-Phe, Tyr, D-Thr, |
| | | L-Dopa, His, D-His, |
| | | Trp, D-Trp, Trans-3,4, |
| | | or 5-phenylproline, cis-3,4, |
| | | or 5-phenylproline |
| Proline | P | D-Pro, L-I-thioazolidine-4- |
| | | carboxylic acid, D-or L- |
| | | 1-oxazolidine-4-carboxylic |
| | | acid |
| Serine | S | D-Ser, Thr, D-Thr, allo-Thr, |
| | | Met, D-Met, Met(O), D-Met(O), |
| | | L-Cys, D-Cys |
| Threonine | T | D-Thr, Ser, D-Ser, allo-Thr, |
| | | Met, D-Met, Met(O), D-Met(O), |
| | | Val, D-Val |
| Tyrosine | Y | D-Tyr, Phe, D-Phe, L-Dopa, |
| | | His, D-His |
| Valine | V | D-Val, Leu, D-Leu, Ile, |
| | | D-Ile, Met, D-Met |

In some embodiments, yeast over-expressing STL1 polypeptides produces at least 0.5%, at least 1%, at least 2%, at least 3%, at least 4%, or even at least 5% more ethanol from a substrate than yeast not overexpressing STL1 polypeptides. In some embodiments, yeast over-expressing STL1 polypeptides produces at least 5%, at least, 10%, at least 11%, at least 12%, at least 13%, at least 14%, or even at least 15% less glycerol from a substrate than yeast not overexpressing STL1 polypeptides. In some embodiments, yeast over-expressing STL1 polypeptides produces at least 5%, at least 10%, at least 20%, at least 25%, at least 30%, at least 35%, at least 40%, or even at least 45% less acetate from a substrate than yeast not over-expressing STL1 polypeptides. In some embodiments, this decrease in acetate is expressly combined with the stated decrease in glycerol and/or increase in ethanol.

The yeast over-expressing STL1 polypeptides additionally expresses either separate phosphoketolase (PKL) and phosphotransacetylase (PTA) polypeptides or PKL-PTA fusion polypeptides. In some embodiments, yeast over-expressing STL1 polypeptides does not have mutations in genes encoding polypeptides in the glycerol synthesis pathway.

In some embodiments, yeast over-expressing STL1 polypeptides expresses the polypeptides at a level that is at least 0.5-fold, 1-fold, 2-fold, 3-fold or greater than yeast not over-expressing STL1 polypeptides, such as the “FG” strain described in the Examples. While the above expression levels refer to protein expression, a convenient way to estimate protein expression levels to measure the amount of mRNA encoding the proteins. In some embodiments, the present modified yeast makes at least 50%, at least 100%, at least 150%, or at least 200% more STL1 mRNA than parental cells, such as the “FG” strain described in the Examples.

An approximately 1-fold increase in expression levels can be achieved by introducing a single copy of an STL1 expression cassette to a cell, the introduced STL1 gene having a promoter of similar strength to the endogenous STL1 promoter of the parental yeast strain. In some embodiments, the promoter is a naturally occurring STL1 promoter. In particular embodiments, the promoter is the same as the endogenous STL1 promoter in the parental yeast strain. An approximately 1-fold increase in expression levels (and mRNA levels) of STL1 can also be achieved by introducing a stronger or regulated promoter into an endogenous STL1 gene or replacing an endogenous STL1 gene with an STL1 expression cassette having a stronger promoter compared to the endogenous STL1 promoter of the parental yeast strain.

### III. Modified Yeast Cells Overexpressing STL1 in Combination with a PKL-PTA Fusion Polypeptide

Engineered yeast cells having a heterologous PKL pathway have been previously described (e.g., WO2015148272). These cells express heterologous PKL (EC 4.1.2.9) and PTA (EC 2.3.1.8), optionally with other enzymes, to channel carbon flux away from the glycerol pathway and toward the synthesis of acetyl-CoA, which is then converted to ethanol. Such modified cells are capable of increased ethanol production in a fermentation process when compared to otherwise-identical parent yeast cells. Unfortunately, such modified also produce increased acetate, which adversely affect cell growth and represents a “waste” of carbon.

Ethanol yield can be increased and acetate production reduced by engineering yeast cells to produce a bi-functional PKL-PTA fusion polypeptide, which includes active portions of both enzymes. Over-expression of such bi-functional fusion polypeptides increases ethanol yield while reducing acetate production by greater than 30% compared to the over-expression of the separate enzymes. It is believed that the expression of separate heterologous PKL and PTA enzymes in a yeast cell allows the production of the intermediate glyceraldehyde-3-phosphate (G-3-P) and acetyl-phosphate (Acetyl-P), the latter being converted to unwanted acetate by an endogenous promiscuous glycerol-3-phosphatase with acetyl-phosphatase activity (GPP1/RHR2). However, by expressing a bi-functional PKL-PTA fusion polypeptide, acetyl-phosphate is rapidly converted to acetyl-CoA, reducing the accumulation of acetyl-phosphate, thereby reducing acetate production. Accordingly, the fusion protein provides a mechanism for the efficient conversion of fructose-6-P (F-6-P) and/or xylulose-5-P (X-5-P) to acetyl-CoA.

The experimental data described, herein, demonstrate that over-expression of STL1 in yeast expressing a PKL-PTA fusion polypeptide further reduces the amount of excess acetate produced from by the PKL pathway. Over-expression of STL1 in yeast also reduced acetate production in yeast expressing PKL and PTA as individual polypeptides.

An exemplary PKL, for expression individually or as a fusion polypeptide, can be obtained from Gardnerella vaginalis (UniProt/TrEMBL Accession No.: WP_016786789) and an exemplary PTA, for expression individually or as a fusion polypeptide, can be obtained from Lactobacillus plantarum (UniProt/TrEMBL Accession No.: WP_003641060). Corresponding enzymes from other organisms are expected to be compatible with the present compositions and methods.

Polypeptides having at least 70%, at least 80%, at least 90%, at least 95%, or more amino acid to the aforementioned PKL and PTA, as well as structural and functional homologs and conservative mutations as exemplified in Table 1, are also expected to be compatible with the present compositions and methods.

### IV. Additional Mutations that Affect Alcohol Production

The present modified cells may further include, or may expressly exclude, mutations that result in attenuation of the native glycerol biosynthesis pathway, which are known to increase alcohol production. Methods for attenuation of the glycerol biosynthesis pathway in yeast are known and include reduction or elimination of endogenous NAD-dependent glycerol 3-phosphate dehydrogenase (GPD) or glycerol phosphate phosphatase activity (GPP), for example by disruption of one or more of the genes GPD1, GPD2, GPP1 and/or GPP2. See, e.g., U.S. Pat. Nos. 9,175,270 (Elke et al.), 8,795,998 (Pronk et al.) and 8,956,851 (Argyros et al.).

The modified yeast may further feature increased acetyl-CoA synthase (also referred to acetyl-CoA ligase) activity (EC 6.2.1.1) to scavenge (i.e., capture) acetate produced by chemical or enzymatic hydrolysis of acetyl-phosphate (or present in the culture medium of the yeast for any other reason) and converts it to acetyl-CoA. This avoids the undesirable effect of acetate on the growth of yeast cells and may further contribute to an improvement in alcohol yield. Increasing acetyl-CoA synthase activity may be accomplished by introducing a heterologous acetyl-CoA synthase gene into cells, increasing the expression of an endogenous acetyl-CoA synthase gene and the like. A particularly useful acetyl-CoA synthase for introduction into cells can be obtained from Methanosaeta concilii (UniProt/TrEMBL Accession No.: WP_013718460). Homologs of this enzymes, including enzymes having at least 85%, at least 90%, at least 92%, at least 95%, at least 97%, at least 98% and even at least 99% amino acid sequence identity to the aforementioned acetyl-CoA synthase from Methanosaeta concilii, are also useful in the present compositions and methods. In other embodiments, the present modified yeast do not have increased acetyl-CoA synthase.

In some embodiments the present modified cells may further include a heterologous gene encoding a protein with NAD+-dependent acetylating acetaldehyde dehydrogenase activity and/or a heterologous gene encoding a pyruvate-formate lyase. The introduction of such genes in combination with attenuation of the glycerol pathway is described, e.g., in U.S. Pat. No. 8,795,998 (Pronk et al.). However, in most embodiments of the present compositions and methods, the introduction of an acetylating acetaldehyde dehydrogenase and/or a pyruvate-formate lyase is not required because the need for these activities is obviated by the attenuation of the native biosynthetic pathway for making acetyl-CoA that contributes to redox cofactor imbalance. Accordingly, in some embodiments, the present yeast do not have a heterologous gene encoding an NAD+-dependent acetylating acetaldehyde dehydrogenase and/or encoding a pyruvate-formate lyase.

In some embodiments, the present modified yeast cells further comprise a butanol biosynthetic pathway. In some embodiments, the butanol biosynthetic pathway is an isobutanol biosynthetic pathway. In some embodiments, the isobutanol biosynthetic pathway comprises a polynucleotide encoding a polypeptide that catalyzes a substrate to product conversion selected from the group consisting of: (a) pyruvate to acetolactate; (b) acetolactate to 2,3-dihydroxyisovalerate; (c) 2,3-dihydroxyisovalerate to 2-ketoisovalerate; (d) 2-ketoisovalerate to isobutyraldehyde; and (e) isobutyraldehyde to isobutanol. In some embodiments, the isobutanol biosynthetic pathway comprises polynucleotides encoding polypeptides having acetolactate synthase, keto acid reductoisomerase, dihydroxy acid dehydratase, ketoisovalerate decarboxylase, and alcohol dehydrogenase activity.

In some embodiments, the modified yeast cells comprising a butanol biosynthetic pathway further comprise a modification in a polynucleotide encoding a polypeptide having pyruvate decarboxylase activity. In some embodiments, the yeast cells comprise a deletion, mutation, and/or substitution in an endogenous polynucleotide encoding a polypeptide having pyruvate decarboxylase activity. In some embodiments, the polypeptide having pyruvate decarboxylase activity is selected from the group consisting of: PDC1, PDC5, PDC6, and combinations thereof. In some embodiments, the yeast cells further comprise a deletion, mutation, and/or substitution in one or more endogenous polynucleotides encoding FRA2, ALD6, ADH1, GPD2, BDH1, and YMR226C. In other embodiments, the present modified yeast cells do not further comprise a butanol biosynthetic pathway.

In some embodiments, the present modified cells include any number of additional genes of interest encoding protein of interest, including selectable markers, carbohydrate-processing enzymes, and other commercially-relevant polypeptides, including but not limited to an enzyme selected from the group consisting of a dehydrogenase, a transketolase, a phosphoketolase, a transladolase, an epimerase, a phytase, a xylanase, a β-glucanase, a phosphatase, a protease, an α-amylase, a β-amylase, a glucoamylase, a pullulanase, an isoamylase, a cellulase, a trehalase, a lipase, a pectinase, a polyesterase, a cutinase, an oxidase, a transferase, a reductase, a hemicellulase, a mannanase, an esterase, an isomerase, a pectinases, a lactase, a peroxidase and a laccase. Proteins of interest may be secreted, glycosylated, and otherwise modified.

### V. Use of the Modified Yeast for Increased Alcohol Production

The present compositions and methods include methods for increasing alcohol production using the modified yeast in fermentation reactions. Such methods are not limited to a particular fermentation process. The present engineered yeast is expected to be a “drop-in” replacement for convention yeast in any alcohol fermentation facility. While primarily intended for fuel ethanol production, the present yeast can also be used for the production of potable alcohol, including wine and beer.

### VI. Yeast Cells Suitable for Modification

Yeast is a unicellular eukaryotic microorganism classified as members of the fungus kingdom and includes organisms from the phyla Ascomycota and Basidiomycota. Yeast that can be used for alcohol production include, but are not limited to, Saccharomyces spp., including S. cerevisiae, as well as Kluyveromyces, Lachancea and Schizosaccharomyces spp. Numerous yeast strains are commercially available, many of which have been selected or genetically engineered for desired characteristics, such as high alcohol production, rapid growth rate, and the like. Some yeast has been genetically engineered to produce heterologous enzymes, such as glucoamylase or α-amylase.

### VII. Substrates and Products

Alcohol production from a number of carbohydrate substrates, including but not limited to corn starch, sugar cane, cassava, and molasses, is well known, as are innumerable variations and improvements to enzymatic and chemical conditions and mechanical processes. The present compositions and methods are believed to be fully compatible with such substrates and conditions.

These and other aspects and embodiments of the present strains and methods will be apparent to the skilled person in view of the present description. The following examples are intended to further illustrate, but not limit, the strains and methods.

### EXAMPLES

### Example 1

### Materials and Methods

### Liquefact Preparation:

Liquefact (corn flour slurry) was prepared by adding 600 ppm of urea, 0.124 SAPU/g ds FERMGEN™ (acid fungal protease) 2.5×, 0.33 GAU/g ds of a variant Trichoderma glucoamylase and 1.46 SSC U/g ds of an Aspergillus α-amylase, adjusted to a pH of 4.8.

Serum vial assays:

2 mL of YPD in 24-well plates were inoculated with yeast cells and the cultures allowed to grow overnight to an OD between 25-30. 2.5 mL liquefact was transferred to serum vials (Chemglass, Catalog #: CG-4904-01) and yeast was added to each vial to a final OD of about 0.4-0.6. The lids of the vials were installed and punctured with needle (BD, Catalog #305111) for ventilation (to release CO₂), then incubated at 32° C. with shaking at 200 RPM for 65 hours.

### AnKom Assays:

300 μL of concentrated yeast overnight culture [this may require more explanation] was added to each of a number ANKOM bottles filled with 50 g prepared liquefact (see above) to a final OD of 0.3. The bottles were then incubated at 32° C. with shaking at 150 RPM for 65 hours.

HPLC analysis:

Samples of the cultures from serum vials and AnKom assays were collected in Eppendorf tubes by centrifugation for 12 minutes at 14,000 RPM. The supernatants were filtered using 0.2 μM PTFE filters and then used for HPLC (Agilent Technologies 1200 series) analysis with the following conditions: Bio-Rad Aminex HPX-87H columns, running temperature of 55° C. 0.6 ml/min isocratic flow 0.01 N H₂SO₄, 2.5 μl injection volume. Calibration standards were used for quantification of the of acetate, ethanol, glycerol, and glucose. The values are expressed in g/L.

### Example 2

### Constructs for Over-Expression of STL1

STL1 from S. cerevisiae and Z. rouxii were codon optimized to generate the coding sequence ScSTLs encoding the polypeptide ScSTLs and the coding sequence ZrSTLs, encoding the polypeptide ZrSTLs, respectively:

SEQ ID NO 1: polynucleotide sequence of the codon-optimized
ScSTLs gene
ATGAAGGACTTGAAGTTGTCTAACTTTAAGGGTAAATTCATCTCCAGAACCTCTCACTGGGG
TTTGACTGGCAAGAAATTGAGATACTTTATCACCATTGCTTCTATGACTGGTTTCTCCTTGT
TTGGTTACGACCAAGGTTTGATGGCTTCTCTAATCACTGGCAAGCAATTCAACTACGAATTT
CCAGCCACCAAGGAAAACGGTGATCACGACAGACATGCTACCGTCGTTCAAGGTGCTACTAC
CTCCTGTTACGAATTGGGTTGTTTTGCTGGTTCTTTGTTCGTCATGTTTTGCGGCGAAAGAA
TCGGTAGAAAGCCATTGATICTAATGGGTICCGTTATCACCATTATCGGIGCTGTCATCTCT
ACTTGTGCCTTTCGTGGTTACTGGGCTTTGGGTCAATTCATCATTGGCAGAGTTGTCACTGG
TGTTGGAACTGGCTTGAACACCTCTACTATTCCAGTCTGGCAATCCGAAATGAGCAAGGCCG
AGAACAGAGGTTTGCTAGTCAACTTGGAAGGTTCTACTATCGCTTTTGGTACCATGATTGCT
TACTGGATCGACTTTGGCTTGTCCTACACCAACAGTTCTGTCCAATGGAGATTTCCAGTTTC
CATGCAAATCGTCTTTGCTTTGTTCTTATTGGCCTTTATGATCAAGTTGCCAGAATCTCCTC
GTTGGTTGATTTCTCAAAGTCGTACCGAAGAGGCTAGATACTTGGTAGGTACTTTAGACGAT
GCCGACCCAAACGATGAAGAGGTCATCACCGAAGTTGCTATGTTGCACGACGCTGTCAACAG
AACCAAGCACGAAAAGCATTCTTTATCCAGCTTGTTCTCCAGAGGTAGGTCTCAAAACTTGC
AGAGAGCTTTGATTGCCGCTTCTACTCAATTCTTTCAGCAATTTACTGGTTGCAACGCTGCC
ATCTACTATTCTACTGTCTTGTTCAACAAGACCATCAAGTTGGACTACAGATTATCTATGAT
CATTGGTGGCGTCTTTGCCACTATCTACGCTTTGTCCACCATCGGTTCTTTCTTTCTAATCG
AAAAGTTGGGTAGACGTAAGCTGTTTTTGTTAGGTGCTACTGGCCAAGCTGTTTCCTTCACC
ATCACTTTTGCCTGTTTGGTCAAGGAAAACAAGGAGAATGCTAGAGGTGCCGCTGTTGGTTT
GTTCCTGTTTATCACCTTCTTTGGTTTGTCTTTACTATCCTTGCCTTGGATCTACCCACCCG
AAATTGCTTCTATGAAGGTTCGTGCCTCCACCAACGCTTTCTCTACTTGTACCAATTGGTTG
TGCAACTTTGCTGTTGTCATGTTTACTCCAATCTTCATTGGTCAATCTGGCTGGGGTTGTTA
CTTGTTCTTTGCCGTTATGAATTACTTGTACATTCCAGTCATCTTCTTTTTCTACCCAGAAA
CTGCTGGTAGAAGCTTGGAGGAAATCGACATTATCTTTGCCAAGGCTTACGAAGATGGTACT
CAACCTTGGAGAGTTGCTAACCACTTACCAAAGTTGTCCTTGCAAGAAGTCGAGGACCACGC
CAACGCTTTGGGTTCTTACGACGATGAAATGGAGAAGGAAGACTTTGGTGAAGACAGAGTCG
AAGATACCTACAACCAAATCAATGGTGACAACTCTTCCAGTTCTTCCAACATCAAGAATGAA
GATACTGTCAACGACAAGGCCAACTTTGAAGGTTAA
SEQ ID NO 2: amino acid sequence of ScSTLs
MKDLKLSNFKGKFISRTSHWGLTGKKLRYFITIASMTGFSLFGYDQGLMASLITGKQFNYEF
PATKENGDHDRHATVVQGATTSCYELGCFAGSLFVMFCGERIGRKPLILMGSVITIIGAVIS
TCAFRGYWALGQFIIGRVVTGVGTGLNTSTIPVWQSEMSKAENRGLLVNLEGSTIAFGTMIA
YWIDFGLSYTNSSVQWRFPVSMQIVFALFLLAFMIKLPESPRWLISQSRTEEARYLVGTLDD
ADPNDEEVITEVAMLHDAVNRTKHEKHSLSSLFSRGRSQNLQRALIAASTQFFQQFTGCNAA
IYYSTVLFNKTIKLDYRLSMIIGGVFATIYALSTIGSFFLIEKLGRRKLFLLGATGQAVSFT
ITFACLVKENKENARGAAVGLFLFITFFGLSLLSLPWIYPPEIASMKVRASTNAFSTCTNWL
CNFAVVMFTPIFIGQSGWGCYLFFAVMNYLYIPVIFFFYPETAGRSLEEIDIIFAKAYEDGT
QPWRVANHLPKLSLQEVEDHANALGSYDDEMEKEDFGEDRVEDTYNQINGDNSSSSSNIKNE
DTVNDKANFEG
SEQ ID NO 3: DNA polynucleotide of the
codon-optimized ZrSTLs gene
ATGGGTAAGAGAACTCAAGGTTTCATGGACTACGTCTTTTCTAGAACCTCCACTGCTGGTTT
GAAGGGTGCTAGATTGCGTTACACTGCTGCCGCTGTTGCCGTCATCGGCTTTGCTTTGTTCG
GTTACGACCAAGGTTTGATGTCTGGTCTAATCACTGGTGATCAATTCAACAAGGAATTTCCA
CCTACCAAGTCCAACGGTGACAATGATCGTTACGCTTCTGTCATTCAAGGTGCCGTTACTGC
TTGTTACGAAATCGGCTGCTTCTTTGGTTCCTTGTTTGTCCTATTCTTTGGTGACGCTATCG
GTAGAAAGCCATTGATCATTTTCGGTGCTATCATTGTCATCATTGGTACCGTTATCTCTACT
GCACCATTTCACCATGCTTGGGGTTTGGGCCAATTCGTTGTCGGTAGAGTTATTACTGGTGT
TGGTACAGGTTTCAACACTTCTACCATTCCAGTGTGGCAATCTGAAATGACGAAACCAAACA
TCAGAGGTGCCATGATCAACTTGGACGGTTCTGTCATTGCTTTTGGTACTATGATCGCTTAC
TGGTTGGACTTCGGCTTTTCCTTCATCAACTCTAGTGTTCAATGGAGATTTCCAGTCTCTGT
TCAAATCATTTTTGCCTTAGTCTTGCTATTCGGTATCGTCAGAATGCCAGAATCTCCCAGAT
GGTTGATGGCCAAGAAAAGACCAGCAGAAGCTAGATACGTGTTGGCTTGTTTGAATGACTTA
CCAGAAAACGACGATGCCATCTTGGCTGAAATGACTTCTTTGCACGAAGCTGTCAACAGATC
CTCTAACCAAAAGTCTCAATGAAGTCCTTGTTCTCTATGGGTAAGCAACAGAACTTTTCCA
GAGCCTTGATTGCTTCTTCCACTCAATTCTTTCAGCAATTCACTGGTTGCAATGCTGCCATC
TACTATTCTACCGTCTTGTTTCAAACCACCGTTCAATTGGACAGATTACTAGCTATGATTTT
GGGTGGCGTCTTTGCCACTGTTTACACCTTGTCTACTTTGCCATCCTTCTACTTAGTCGAAA
AGGTTGGTAGACGTAAGATGTTTTTCTTTGGTGCTTTGGGTCAAGGCATCTCCTTCATCATT
ACATTTGCTTGTTTGGTCAATCCAACCAAGCAAAACGCCAAGGGTGCTGCCGTTGGTTTGTA
CTTATTCATCATTTGTTTTGGTTTGGCTATCTTAGAATTGCCTTGGATCTACCCACCTGAAA
TTGCTTCTATGAGAGTTCGTGCAGCTACCAACGCCATGTCTACCTGTACTAACTGGGTTACC
AACTTTGCTGTTGTTATGTTCACTCCAGTCTTCATCCAAACTTCTCAATGGGGTTGTTACTT
GTTCTTTGCTGTTATGAACTTCATCTACTTGCCAGTTATCTTTTTCTTTTACCCAGAAACTG
CTGGTAGATCCTTGGAAGAGATCGACATTATCTTTGCCAAGGCTCACGTGGACGGTACCTTG
CCTTGGATGGTTGCTCACAGATTACCAAAGTTGTCTATGACCGAAGTTGAGGACTACTCCCA
ATCTTTGGGTCTACACGATGACGAAAACGAAAAGGAGGAATACGACGAGAAGGAAGCTGAAG
CCAATGCTGCCTTGTTTCAAGTCGAAACTTCTTCCAAGTCTCCATCCTCTAACAGAAAGGAC
GATGACGCTCCAATCGAACATAACGAGGTTCAAGAATCCAACGACAATTCTTCCAACAGCTC
TAACGTCGAAGCTCCAATTCCTGTTCATCACAACGATCCATAA
SEQ ID NO 4: amino acid sequence of ZrSTLs
MGKRTQGFMDYVFSRTSTAGLKGARLRYTAAAVAVIGFALFGYDQGLMSGLITGDQFNKEFP
PTKSNGDNDRYASVIQGAVTACYEIGCFFGSLFVLFFGDAIGRKPLIIFGAIIVIIGTVIST
APFHHAWGLGQFVVGRVITGVGTGFNTSTIPVWQSEMTKPNIRGAMINLDGSVIAFGTMIAY
WLDFGFSFINSSVQWRFPVSVQIIFALVLLFGIVRMPESPRWLMAKKRPAEARYVLACLNDL
PENDDAILAEMTSLHEAVNRSSNQKSQMKSLFSMGKQQNFSRALIASSTQFFQQFTGCNAAI
YYSTVLFQTTVQLDRLLAMILGGVFATVYTLSTLPSFYLVEKVGRRKMFFFGALGQGISFII
TFACLVNPTKQNAKGAAVGLYLFIICFGLAILELPWIYPPEIASMRVRAATNAMSTCTNWVT
NFAVVMFTPVFIQTSQWGCYLFFAVMNFIYLPVIFFFYPETAGRSLEEIDIIFAKAHVDGTL
PWMVAHRLPKLSMTEVEDYSQSLGLHDDENEKEEYDEKEAEANAALFQVETSSKSPSSNRKD
DDAPIEHNEVQESNDNSSNSSNVEAPIPVHHNDP

Expression vector pZK41Wn was used to express the codon optimized STL1 polypeptides. The starting plasmid lacks an expression cassette and is designed to integrate a 389-bp synthetic DNA fragment with multiple endonuclease restriction sites into the Saccharomyces chromosome downstream of YHL041W locus.

Plasmid pK41Wn-DScSTL contains a cassette to express ScSTLs under the control of the promoter of the gene encoding cytosolic copper-zinc superoxide dismutase (SOD1; and the terminator of the gene encoding 3-phosphoglycerate kinase (PGK1).

SEQ ID NO 5: polynucleotide sequence of the
SOD1 promoter
GTCAAAAATAGCCATCTTAGCATCGCCTGATTTGGCATCGACC
AAAATTGCGTCGTTTTCCTTTAGAGAATACTTGGCCAGGTATT
CAGCCGTGACGTCGGCTTGGAAATCTAAAAGTGGGTTACCCAA
TACTACCAATGGTGCGGTCATAATTGCTTGCTCTTTCTTTTGC
TGTTATCTTTGGTTCTACCCTGCACAAGATAAACTGAGATGAC
TACCTAATTAGACATGGCATGCCTATAAGTAAAGAGAATTGGG
CTCGAAGAATAATTTTCAAGCCTGCCCTCATCACGTACGACGA
CACTGCGACTCATCCATGTGAAAATTATCGGCATCTGCAAAAA
AAGTTTCAACTTCCACAGGTAATATTGGCATGATGCGAAATTG
GACGTAAGTATCTCTGAAGTGCAGCCGATTGGGCGTGCGACTC
ACCCACTCAGGACATGATCTCAGTAGCGGGTTCGATAAGGCGA
TGACAGCGCAAATGCCGCTTACTGGAAGTACAGAACCCGCTCC
CTTAGGGGCACCCACCCCAGCACGCCGGGGGGTTAAACCGGTG
TGTCGGAATTAGTAAGCGGACATCCCTTCCGCTGGGCTCGCCA
TCGCAGATATATATATAAGAAGATGGTTTTGGGCAAATGTTTA
GCTGTAACTATGTTGCGGAAAAACAGGCAAGAAAGCAATCGCG
CAAACAAATAAAACATAATTATTTAT

Plasmid pZK41Wn-DScSTL is designed to integrate the SOD 1::ScSTLs::PGK1 expression cassette into the Saccharomyces chromosome downstream of YHL041W locus. The functional and structural composition of plasmid pZK41Wn-DScSTL is described in Table 3.

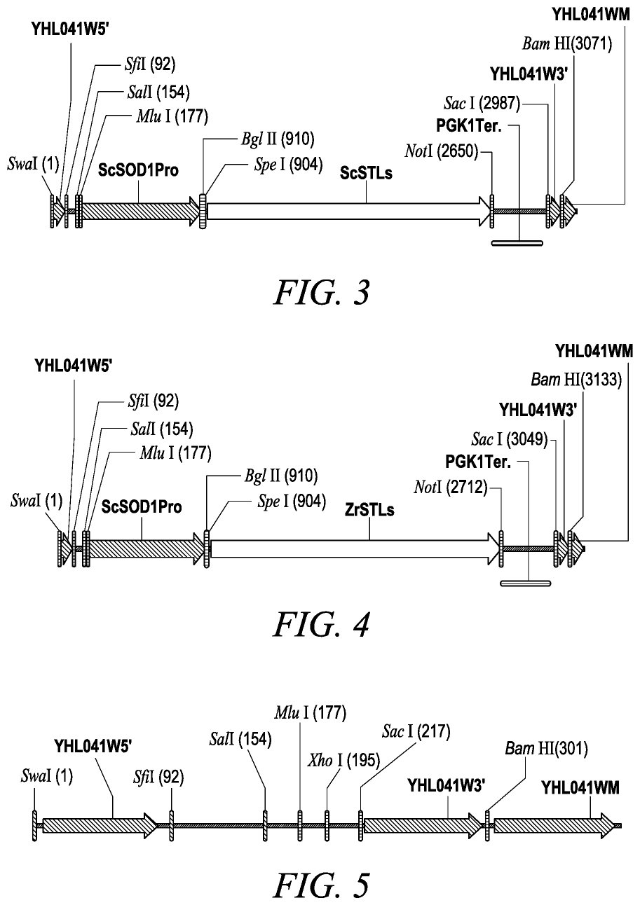

TABLE 3
Functional and structural elements of plasmid pZK41Wn-DScSTL

| Functional/structural element | Description |
|---|---|
| “YHL041W3′” fragment, | 78-bp DNA fragment (labeled as |
| downstream of YHL041W locus | YHL041W3′ in FIG. 3) from |
| | S. cerevisiae |
| “YHL041WM” fragment, | 80-bp DNA fragment (labeled as |
| downstream of YHL041W locus | YHL041WM in FIG. 3) from |
| | S. cerevisiae |
| ColE1 replicon and ampicillin | These sequences are not part of |
| resistance marker gene | the DNA fragment integrated |
| | into yeast genome |
| “YHL041W5′” fragment, | 76-bp DNA fragment (labeled as |
| downstream of YHL041W locus | YHL041W5′ in FIG. 3) |
| SOD1Promoter:: ScSTLs::PGK1 | Cassette for expression of codon |
| Terminator | optimized ScSTLs |

The structural of pZK41Wn-DZrSTL is parallel to pZK41Wn-DScSTL, except that it contains a cassette to express ZrSTLs instead of ScSTLs. Plasmid pZK41Wn-DZrSTL is designed to integrate the SOD1::ZrSTLs::PGK1 expression cassette into the Saccharomyces chromosome downstream of YHL041W locus.

### Example 3

### Generation of strains G614, G697 & G751 from industrial yeast FERMAX™ Gold

To study the effects of STLs in industrial yeast, the wild-type FERMAX™ Gold strain (Martrex, Inc., Chaska, Minn., USA), hereafter abbreviated, “FG,” was used as a parent to introduce the STLs expression cassettes and control fragment individually. Cells were transformed either (i) a 3,159-bp SwaI fragment containing the SOD1::ScSTLs::PGK1 expression cassette from plasmid pZK41Wn-DScSTL, (ii) a 3,221-bp SwaI fragment containing SOD1::ZrSTLs::PGK1 expression cassette from plasmid pZK41Wn-DZrSTL, or (iii) a 389-bp SwaI fragment containing a synthetic DNA fragment with poly linkers from vector pZK41Wn, using standard methods. Transformants were selected and designated as shown in Table 4.

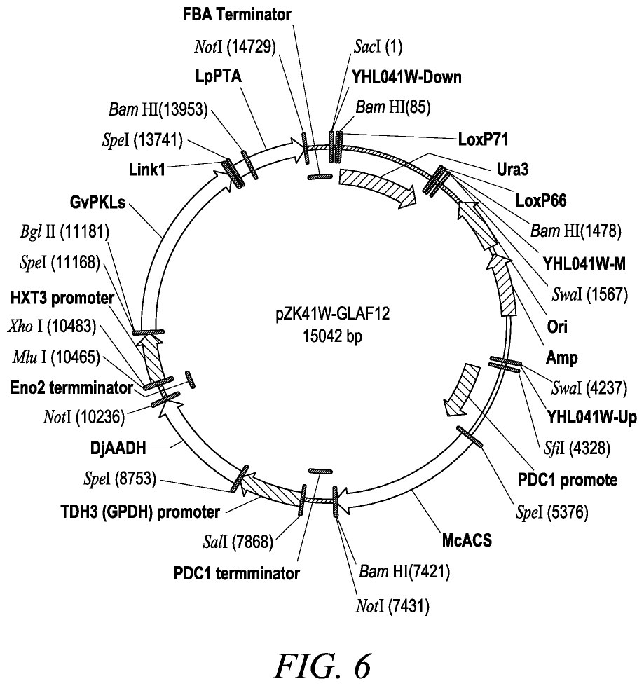

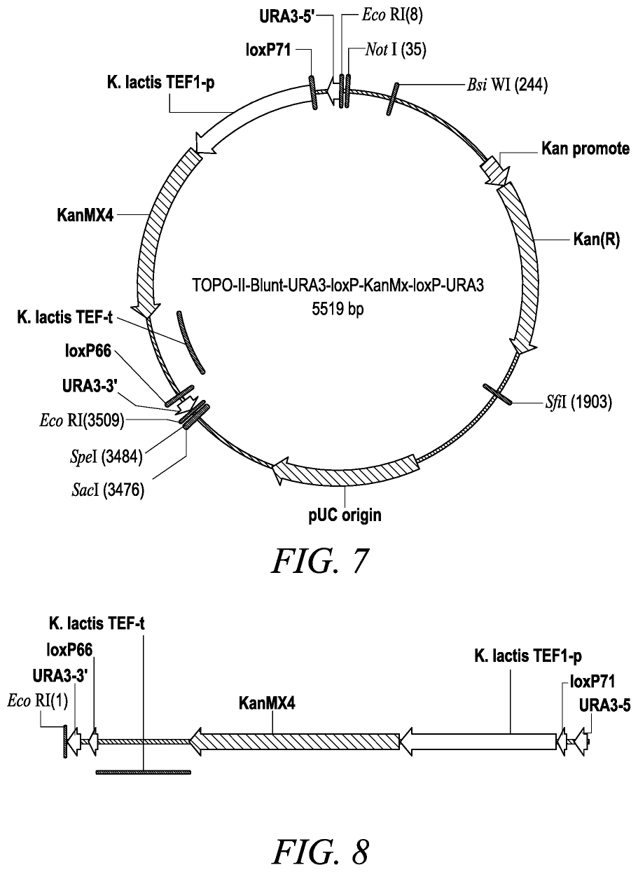

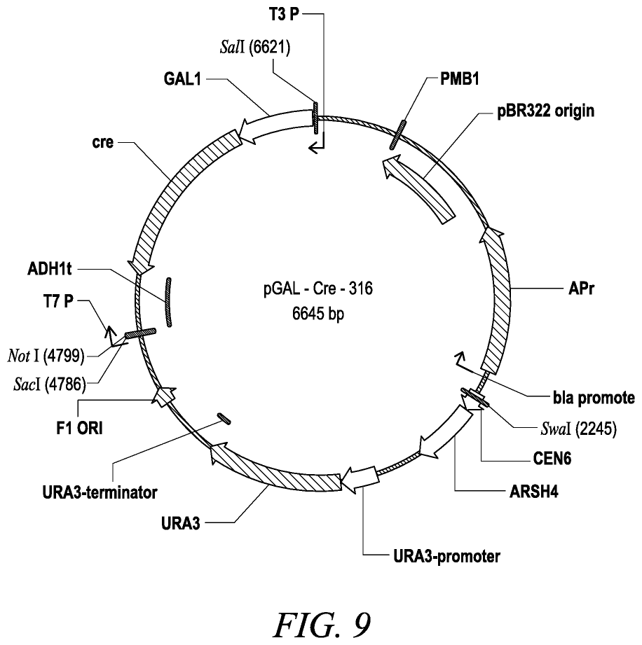

TABLE 4
Designation of selected transformants

| | | Integration | Transgene(s) |
|---|---|---|---|
| Strain | Insert | site | expressed |
| G597 | SwaI fragment from | Downstream of | SOD1::ScSTLs::PGK1 |
| | pZK41Wn-DScSTL | YHL041W | |
| | (FIG. 6) | | |
| G614 | SwaI fragment from | Downstream of | SOD1::ZrSTLs::PGK1 |
| | pZK41Wn-DZrSTL | YHL041W | |
| | (FIG. 7) | | |
| G751 | SwaI fragment from | Downstream of | Synthetic DNA fragment |
| | pZK41Wn (FIG. 8) | YHL041W | with poly-linkers |

Example 4

### Comparison of Strains Expressing Different STLs In Vial Assays

The new FG yeast strains G597, G614 and G751, along with their parent strain, FG, were grown in vial cultures and their fermentation products analyzed as described in Example 1. Performance in terms of ethanol, glycerol and acetate production is shown in Table 5.

TABLE 5
FG versus G597, G614 and G751 in vial assays

| Strain | Transgenet(s) expressed | EtOH | Glycerol | Acetate |
|---|---|---|---|---|
| FG | none | 142.93 | 17.27 | 0.76 |
| G597 | ScSTLs | 143.43 | 14.97 | 0.64 |
| FG | none | 142.93 | 17.27 | 0.76 |
| G614 | ZrSTLs | 144.25 | 14.05 | 0.60 |
| FG | none | 147.83 | 17.12 | 1.10 |
| G751 | none | 147.72 | 17.08 | 1.13 |

The performance of control strain G751 and FG parent are almost identical in terms of the titers of ethanol, glycerol and acetate, demonstrated that the integration of the synthetic DNA fragment at the downstream of YHL041W locus did not affect the ethanol production. G597 and G614 yeast that over-expressed ScSTLs or ZrSTLs, respectively, produced slightly more ethanol and significantly less glycerol and acetate than the FG parent or strain G751 with the control DNA fragment.

### Example 5

### Further Comparison of Strains Expressing STLs in AnKom assays

To confirm the benefits of over-expressing ScSTLs and ZrSTLs, the performance of strains G597 and G614 were more precisely analyzed in better-controlled AnKom assays, as described in Example 1. Performance in terms of ethanol, glycerol and acetate production is shown in Table 6.

TABLE 6
FG versus G597 and G614 in AnKom assays

| Strain | Transgene(s) expressed | EtOH | Glycerol | Acetate |
|---|---|---|---|---|
| FG | none | 139.32 | 15.62 | 0.81 |
| G597 | ScSTLs | 140.80 | 13.32 | 0.52 |
| G614 | ZrSTLs | 142.52 | 12.59 | 0.47 |

The increase in ethanol production with strains G597 and G614 was about 1.1% and 2.3%, respectively, compared to the FG parent strain. The reduction of glycerol with the strains G597 and G614 was 14.7% and 19.4%, respectively compared to the FG parent strain. Most surprising was that acetate reduction with strains G597 and G614 was 35.8% and 42.0%, respectively, compared to the FG parent strain.

Example 6

### Plasmid pZK41W-GLAF12 with Phosphoketolase-Phosphotransacetylase Fusion Gene 1

Synthetic phosphoketolase and phosphotransacetylase fusion gene 1, GvPKL-L1-LpPTA, includes the codon-optimized coding regions for the phosphoketolase from Gardnerella vaginalis (GvPKL) and the phosphotransacetylase from Lactobacillus plantarum (LpPTA) joined with a synthetic linker. The amino acid sequence of the fusion polypeptide, with the linker region shown in bold italics, is shown as SEQ ID NO: 6.

SEQ ID NO 6: amino acid sequence of the
GvPKL-L1&LpPTA fusion protein
MTSPVIGTPWKKLNAPVSEAAIEGVDKYWRVANYLSIGQ
IYLRSNPLMKEPFTREDVKHRLVGHWGTTPGLNFLIGHI
NRFIAEHQQNTVIIMGPGHGGPAGTAQSYLDGTYTEYYP
KITKDEAGLQKFFRQFSYPGGIPSHFAPETPGSIHEGGE
LGYALSHAYGAVMNNPSLFVPAIVGDGEAETGPLATGWQ
SNKLVNPRTDGIVLPILHLNGYKIANPTILSRISDEELH
EFFHGMGYEPYEFVAGFDDEDHMSIHRRFADMFETIFDE
ICDIKAEAQTNDVTRPFYPMIIFRTPKGWTCPKFIDGKK
TEGSWRAHQVPLASARDTEAHFEVLKNWLKSYKPEELFN
EDGSIKEDVLSFMPQGELRIGQNPNANGGRIREDLKLPN
LDDYEVKEVKEFGHGWGQLEATRRLGVYTRDVIKNNPDS
FRIFGPDETASNRLQAAYEVTNKQWDAGYLSELVDEHMA
VTGQVTEQLSEHQMEGFLEAYLLTGRHGIWSSYESFVHV
IDSMLNQHAKWLEATVREIPWRKPISSMNLLVSSHVWR
QDHNGFSHQDPGVTSVLLNKTFNNDHVIGIYFPVDSNML
LAVGEKVYKSTNMINAIFAGKQPAATWLTLDEAREELEK
GAAEWKWASNAKNNDEVQVVLAGIGDVPQQELMAAADKL
NKLGVKFKVVNIVDLLKLQSAKENNEALTDEEFTELFTA
DKPVLLAYHSYAHDVRGLIFDRPNHDNFNVHGYKEQGST
TTPYDMVRVNDMDRYELTAEALRMVDADKYADEIKKLED
FRLEAFQFAVDKGYDHPDYTDWVWPGVKTDKPGAVTATA
ATAGDNE [symbol] [symbol] MDLFESLAQ
KITGKDQTIVFPEGTEPRIVGAAARLAADGLVKPIVLGA
TDKVQAVANDLNADLTGVQVLDPATYPAEDKQAMLDALV
ERRKGKNTPEQAAKMLEDENYFGTMLVYMGKADGMVSGA
IHPTGDTVRPALQIIKTKPGSHRISGAFIMQKGEERYVF
ADCAINIDPDADTLAEIATQSAATAKVFDIDPKVAMLSF
STKGSAKGEMVTKVQEATAKAQAAEPELAIDGELQFDAA
FVEKVGLQKAPGSKVAGHANVFVFPELQSGNIGYKIAQR
FGHFEAVGPVLQGLNKPVSDLSRGCSEEDVYKVAIITAA
QGLA

Plasmid pZK41W-GLAF12 contains three cassettes to express the GvPKL-L1-LpPTA fusion polypeptide, acylating acetaldehyde dehydrogenase from Desulfospira joergensenii (DjAADH), and acetyl-CoA synthase from Methanosaeta concilii (McACS). Both DjAADH and McACS were codon optimized. The expression of GvPKL-L1-LpPTA is under the control of an HXT3 promoter and FBA1 terminator. The expression of DjAADH is under the control of TDH3 promoter and ENO2 terminator. The expression of McACS is under the control of PDC1 promoter and PDC1 terminator. Plasmid pZK41W-GLAF12 was designed to integrate the three expression cassettes into the Saccharomyces chromosome downstream of the YHL041W locus. The functional and structural composition of plasmid pZK41W-GLAF12 is described in Table 7.

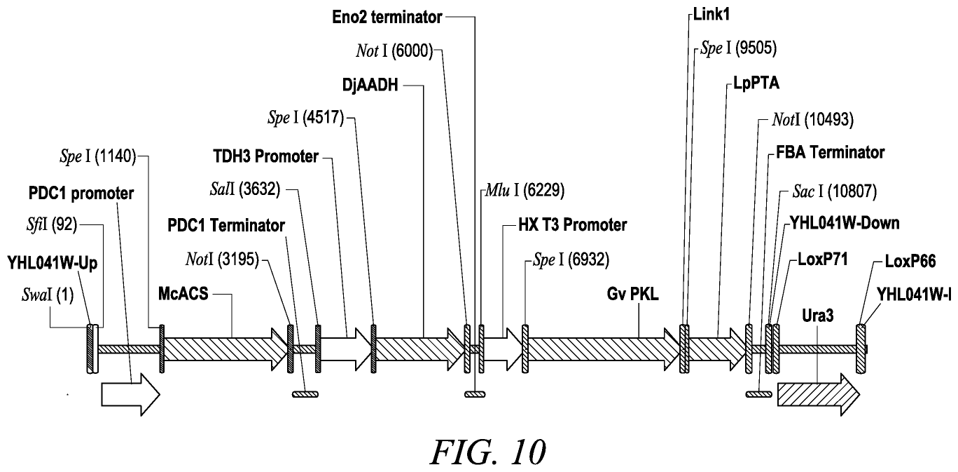

TABLE 7
Functional and structural elements of plasmid pZK41W-GLAF12

| Functional/Structural element | Description |
|---|---|
| “Down” fragment, downstream | 78-bp DNA fragment (labeled as YHL041W- |
| of YHL041W locus | Down in FIG. 10) from S. cerevisiae |
| LoxP71 site | LoxP71 site |
| Ura3 gene | Ura3 gene used as selection marker |
| LoxP66 | LoxP66 site |
| “M” fragment, downstream of | 80-bp DNA fragment (labeled as YHL041W-M |
| YHL041W locus | in FIG. 10) from S. cerevisiae |
| ColE1 replicon and ampicillin | These sequences are not part of the DNA |
| resistance marker gene | fragment integrated into yeast genome |
| “Up” fragment, downstream of | 76-bp DNA fragment (labeled as |
| YHL041W locus | YHL041W-Up in FIG. 10) |
| PDC1Promoter::McACS::PDC | Cassette for expression of codon optimized |
| Terminator | McACS encoding acetyl-CoA synthase, derived |
| | from M. consilii |
| TDH3 Promoter::DjAADH::ENO | Cassette for expression of codon optimized |
| Terminator | DjAADH encoding acylating acetaldehyde |
| | dehydrogenase, derived from D. joergensenii |
| HXT3 Promoter::GvPKL-L1- | Cassette for expression of codon-optimized |
| LpPTA::FBA1 Terminator. | GvPKL-L1-LpPTA fusion gene |

### Example 7

### Generation an FG-ura3 Strain with a ura3 Genotype

The FG strain was used as the “wild-type” parent strain to make the ura3 auxotrophic strain FG-ura3. Plasmid pTOPO II-Blunt ura3-loxP-KanMX-loxP-ura3 was designed to replace the URA3 gene in strain FG with mutated ura3 and URA3-loxP-TEFp-KanMX-TEFt-loxP-URA3 fragment. The functional and structural elements of the plasmid are listed in Table 8.

TABLE 8
Functional/structural elements of pTOPO
II-Blunt ura3-loxP-KanMX-loxP-ura3

| Functional/Structural Element | Comment |
|---|---|
| KanR gene in E. coli | Vector sequence |
| pUC origin | Vector sequence |
| URA3 3′-flanking region, | Synthetic DNA identical to |
| | S. cerevisiae |
| | genomic sequence to URA3 locus |
| loxP66 | Synthetic DNA identical to loxP66 |
| | consensus |
| TEF1::KanMX4::TEF Terminator | KanMX expression cassette |
| loxP71 | Synthetic DNA identical to loxP71 |
| | consensus |
| URA3 5′-flanking region | Synthetic DNA identical to the URA3 |
| | locus on the S. cerevisiae |
| | genome |

A 2,018-bp DNA fragment containing the ura3-loxP-KanMX-loxP-ura3 cassette was released from plasmid TOPO II-Blunt ura3-loxP-KanMX-loxP-ura3 by EcoRI digestion. The fragment was used to transform S. cerevisiae FG cells by electroporation.

Transformed colonies able to grow on media containing G418 were streaked on synthetic minimal plates containing 20 pg/mluracil and 2 mg/ml 5-fluoroorotic acid (5-FOA). Colonies able to grow on 5-FOA plates were further confirmed for URA3 deletion by growth of phenotype on SD-Ura plates, and by PCR. The ura3 deletion transformants were unable to grow on SD-Ura plates. A single 1.98-kb PCR fragment was obtained with test primers. In contrast, the same primer pairs generated a 1.3-kb fragment using DNA from the parental FG strain, indicating the presence of the intact ura3 gene. The ura3 deletion strain was named as FG-KanMX-ura3.

To remove the KanMX expression cassette from strain FG-KanMX-ura3, plasmid pGAL-Cre-316 was used to transform cells of strain FG-KanMX-ura3 by electroporation. The purpose of using this plasmid is to temporary express the Cre enzyme, so that the LoxP-sandwiched KanMX gene will be removed from strain FG-KanMX-ura3 to generate strain FG-ura3. pGAL-Cre-316 is a self-replicating circular plasmid that was subsequently removed from strain FG-ura3. None of the sequence elements from pGAL-cre-316 was inserted into the strain FG-ura3 genome. The functional and structural elements of plasmid pGAL-Cre-316 is listed in Table 9.

TABLE 9
Functional and structural elements of pGAL-Cre-316.
Functional/Structural element

Yeast-bacterial shuttle vector pRS316 sequence
pBR322 origin of replication
S. cerevisiae URA3 gene
F1 origin
GALp-Cre-ADHt cassette, reverse orientation

The transformed cells were plated on SD-Ura plates. Single colonies were transferred onto a YPG plate and incubated for 2 to 3 days at 30° C. Colonies were then transferred to a new YPD plate for 2 additional days. Finally, cell suspensions from the YPD plate were spotted on to following plates: YPD, G418 (150 μg/ml), 5-FOA (2 mg/ml) and SD-Ura. Cells able to grow on YPD and 5-FOA, and unable to grow on G418 and SD-Ura plates, were picked for PCR confirmation as described, above. The expected PCR product size was 0.4-kb and confirmed the identity of the KanMX (geneticin)-sensitive, ura3-deletion strain, derived from FG-KanMX-ura3. This strain was named as FG-ura3.

Example 8

### Generation of Strain G176 Expressing PKL and PTA as a Fusion Polypeptide

The FG-ura3 strain was used as a parent to introduce the PKL pathway in which PKL and PTA genes are fused together with linker 1 as described, above. Cells were transformed with a 12,372-bp SwaI fragment containing the GvPKL-L1-LpPTA expression cassette from plasmid pZK41W-GLAF12. One transformant with the SwaI fragment from pZK41W-GLAF12 integrated at the downstream of YHL041W locus was selected and designated as strain G176.

The new FG yeast strains G176 and its parent strain, FG, were grown in vial cultures and their fermentation products analyzed as described in Example 1. Performance in terms of ethanol, glycerol and acetate production is shown in Table 10.

TABLE 10
FG versus G176 in vial assays

| Strain | Transgene(s) expressed | EtOH | Glycerol | Acetate |
|---|---|---|---|---|
| FG | none | 131.89 | 16.30 | 0.60 |
| G176 | GvPKL-L1-LpPTA fusion | 142.15 | 13.95 | 1.10 |

Strain G176 produced more ethanol and less glycerol than the FG parent, which is desirable in terms of performance. Strain G176 produced more acetate than the FG parent.

To confirm the performance of strain G176, FG and G176 strains were more precisely analyzed in better-controlled AnKom assays, as described in Example 1. Performance in terms of ethanol, glycerol and acetate production is shown in Table 11.

TABLE 11
FG versus G176 in AnKom assays

| Strain | Transgene(s) expressed | EtOH | Glycerol | Acetate |
|---|---|---|---|---|
| FG | none | 135.52 | 16.68 | 0.79 |
| G176 | GvPKL-L1-LpPTA fusion | 143.92 | 14.70 | 1.29 |

The increase in ethanol production with the G176 was 6.2% of its parent FG; the decrease in glycerol production was 11.9% of its parent FG. The increase in acetate production was 63.30% of its parent FG, which was not a desirable trait of the ethanol production strain for industrial applications.

### Example 9

### Generation of Strains G709, G569 and G711 from G176

With reference to the previous Examples, the codon-optimized STL1 from S. cerevisiae and Z. rouxii were introduced into the G176 strain. Expression vector pZKH1 is similar to pZK41Wn except that it is designed as a to integrate at the Saccharomyces chromosome downstream of hexose transporter 1 gene (HXT1, YHR094C locus). As in Example 2, plasmids were made to express ScSTLs or ZrSTLs under the control of the promoter of SOD1 and the terminator of PGK1. Transformants were selected and designated as shown in Table 12.

TABLE 12
Designation of selected transformants

| | | Integration | |
|---|---|---|---|
| Strain | Insert | site | Transgene(s) expressed |
| G709 | SwaI fragment | Downstream of | Synthetic DNA fragment with |
| | from pZKH1 | YHR094C | poly-linkers and GvPKL-L1- |
| | (FIG. 19) | locus | LpPTA fusion from G176 |
| G569 | SwaI fragment | Downstream of | SOD1::ScSTLs::PGK1 and |
| | from pZKH1- | YHR094C | GvPKL-L1-LpPTA fusion |
| | DScSTL | locus | from G176 |
| | (FIG. 20) | | |
| G711 | SwaI fragment | Downstream of | SOD1::ZrSTLs::PGK1 and |
| | from pZKH1- | YHR094C | GvPKL-L1-LpPTA fusion |
| | DZrSTL | locus | from G176 |
| | (FIG. 21) | | |

### Example 10

### Comparison of Strains Expressing ScSTLs or ZrSTLs in Vial Assays

The new strains G569, G709 and G711, derived from strain G176, along with the FG strain, were grown in vial cultures and their fermentation products analyzed as described in Example 1. Performance in terms of ethanol, glycerol and acetate production is shown in Table 13.

TABLE 13
FG versus G569, G709 and G711 in vial assays

| Strain | Transgene(s) expressed | EtOH | Glycerol | Acetate |
|---|---|---|---|---|
| FG | none | 140.81 | 16.14 | 0.56 |
| G709 | GvPKL-L1-LpPTA fusion | 142.07 | 14.27 | 1.04 |
| FG | none | 136.17 | 17.00 | 0.76 |
| G569 | ScSTLs, GvPKL-L1-LpPTA fusion | 141.99 | 12.31 | 0.69 |
| FG | none | 140.81 | 16.14 | 0.56 |
| G711 | ZrSTLs, GvPKL-L1-LpPTA fusion | 143.18 | 12.33 | 0.75 |

In comparison to FG yeast, modified G709 yeast that express the PKL-PTA fusion polypeptide produce more ethanol and less glycerol but significantly more acetate. This is consistent with results described in Example 9. However, modified G569 and G711 yeast, which over-express an STL1 in addition to the PKL-PTA fusion polypeptide, while still producing more acetate than FG yeast, produce significantly less addition acetate than yeast that do not over-express an STL1. Modified yeast that over-express an STL1 in addition to expressing separate PKL and PTA polypeptides also produced significantly less addition acetate than yeast that do not over-express an STL1 (data not shown).

### Example 11

### Comparison of Strains Expressing STL1s in AnKom Assays

To confirm the benefits of over-expression ScSTLs and ZrSTLs, the performance of strains G569, G709, G711 and their parent G176 were more precisely analyzed in better-controlled AnKom assays, as described in Example 1. Performance in terms of ethanol, glycerol and acetate production is shown in Table 14.

TABLE 14
G176 versus G569, G709 and G711 in AnKom assays

| Strain | Transgene(s) expressed | EtOH | Glycerol | Acetate |
|---|---|---|---|---|
| G176 | GvPKL-L1-LpPTA fusion | 141.29 | 14.82 | 1.17 |
| G709 | Control fragment, | 141.21 | 14.72 | 1.16 |
| | GvPKL-L1-LpPTA fusion | | | |
| G569 | ScSTLs, | 143.47 | 13.16 | 0.89 |
| | GvPKL-L1-LpPTA fusion | | | |
| G711 | ZrSTLs, | 145.02 | 12.85 | 0.91 |
| | GvPKL-L1-LpPTA fusion | | | |

The performance of strains G709 and parent G176, which both express the PKL-PTA fusion polypeptide, was almost identical, confirming that the integration of the synthetic DNA fragment at the downstream of YHR094C locus did not affect the performance of the yeast in fermentation. The increase in ethanol production with the strains G569 and G711, which over-expression ScSTLs and ZrSTLs, respectively, was 1.5% and 2.6%, respectively, compared to parental strain G176. The reduction of glycerol with strains G569 and G711 was 11.2 and 13.3%, respectively, compared to parental strain G176, respectively. The acetate production with strains G569 and G711 was reduced by 23.9% and 22.2%, respectively, compared to parental strain G176.

The results of this experiment demonstrate that the expression of enzymes in the PKL pathway and over-expression of STLs can be combined to increase ethanol production, while simultaneously reducing the production of glycerol and acetate by-products.

What is claimed is:

1. A method for decreasing the production of glycerol and acetate in cells grown on a carbohydrate substrate, comprising: introducing into modified yeast comprising an exogenous pathway that causes it to produce more ethanol and acetate than its parental yeast a genetic alteration that increases the production of STL1 polypeptides compared to the amount produced in the parental yeast.

2. The method of claim 1, wherein the genetic alteration comprises introducing an expression cassette for expressing an STL1 polypeptide.

3. The method of claim 1, wherein the genetic alteration comprises introducing an exogenous gene encoding an STL1 polypeptide.

4. The method of claim 1, wherein the genetic alteration comprises introducing a stronger or regulated promoter in an endogenous gene encoding an STL1 polypeptide.

5. The method of any of claims 1-4, wherein the decrease in production of acetate is at least 10% compared to the production by the parental cells grown under equivalent conditions.

6. The method of any of claims 1-5, wherein the decrease in production of acetate is at least 15% compared to the production by the parental cells grown under equivalent conditions.

7. The method of any of claims 1-6, wherein the exogenous pathway is the phosphoketolase pathway.

8. The method of claim 7, wherein the phosphoketolase pathway includes a phosphoketolase enzyme and a phosphotransacetylase enzyme.

9. The method of claim 8, wherein the phosphoketolase and phosphotransacetylase are in the form of a fusion polypeptide.

10. The method of any of claims 1-9, wherein the cells further comprise an exogenous gene encoding a carbohydrate processing enzyme.

11. The method of claim 10, wherein the carbohydrate processing enzyme is a glucoamylase or an alpha-amylase.

12. The method of any of claims 1-11, wherein the cells further comprise an alteration in the glycerol pathway and/or the acetyl-CoA pathway.

13. The method of any of claims 1-12, wherein the cells are of a Saccharomyces spp.
